(19) United States
(12) Patent Application Publication
(10) Pub. No.: US 2004/0082896 A1
(43) Pub. Date: Apr. 29, 2004

(54) Apparatus for performing spinal therapy

(76) Inventors: Robert D. Harris, Logan, UT (US); William R. Hachtmann, Oakhurst, CA (US); Daniel Haynie, North Logan, UT (US); Michael Lee Wildman, Logan, UT (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/641,552

(22) Filed: Aug. 14, 2003

Related U.S. Application Data
(63) Continuation-in-part of application No. 10/283,846, filed on Oct. 29, 2002.

(51) Int. Cl. A61F 5/00

(52) U.S. Cl. 602/32

(57) ABSTRACT

An apparatus for performing spinal therapy includes a base assembly having a pair of spaced-apart parallel guide posts extending upwardly from a platform. The guide posts are capable of being pivoted between a first vertical position, and a second angled position for performing different stretches. Two lift bars are slidably attached to the guide posts, with a cross bar interconnected therebetween. A mattress is disposed between the guide posts and over at least a portion of the cross bar. The mattress is reinforced using resiliently flexible slats to cause the mattress to bow while the lift bar assembly is raised.

### RELATED APPLICATION

This application is a Continuation-in-Part of U.S. application Ser. No. 10/283,846, filed Oct. 29, 2002.

### BACKGROUND OF THE INVENTION

The present invention relates to exercise and physical therapy machines. More particularly, the present invention relates to a process for performing spinal therapy using a reinforced mattress in association with an apparatus which provides vertical lifting action of a user's spine in a passive manner.

The spine is comprised of a bony column forming the main structural support of the human skeleton. It consists of vertebrae segments linked by flexible joints and held together by gelatinous discs of cartilage and ligaments. Each vertebrae has a somewhat cylindrical bony body, a number of wing-like projections, and a bony arch. There are twenty-four movable vertebrae, seven cervical, twelve dorsal and five lumbar.

The lumbar and cervical regions of the spine normally define forward curves of about 35 to 45 degrees, whereby weight is distributed relatively evenly on the individual discs within the region. This curvature can be lost due to a variety of causes, including injury from lifting, bad posture, sitting for prolonged periods of time, viewing computer monitors in a “hunched” position, and increased age. When the curvature is lost, uneven and increased pressure develops on a few of the vertebrae and inflammation or restricted fluid flows occurs resulting in back pain and loss of mobility.

It is estimated that between 60% and 80% of the general population will suffer from low back pain at one point in their lives, and that between 20% and 30% of the population are suffering from back pain at any given time. It is also estimated that there are over 13 million annual doctor visits for low back visits, thus being the second most frequently reported reason for visiting a doctor. There are over 50 million annual visits to chiropractors for lower back pain. It is reported that low back pain is the third most frequent reason for surgery, and the second leading cause of absenteeism from work.

An underlying problem with nearly all back pain is the compression of the spinal vertebrae and/or surrounding muscle tension. If left untreated, the uneven weight on the vertebrae can cause intervertebral discs to wear and degenerate, neuralgic problems such as pinched nerves can arise, and calcification and scarring of the spine can occur. Over time, the loss of mobility can cause the spine to lose its curvature and a rounded hump, known as Dowagers Hump, can develop with increased age.

It is generally accepted that proper mobilization and stretching of the spine can alleviate pain and increase range of motion as well as the overall function of the back and body. A number of devices exist which attempt to remedy back pain by stretching and strengthening the back and spine. However, all of these devices suffer certain drawbacks. Some of the devices are active in that the user must move his or her body, sometimes with resistance, in order to attain the stretch or exercise the back muscles. For many who suffer back pain, these motions and accompanying resistance are too rigorous to be performed without pain. This is particularly the case for the elderly. If not done correctly, these exercises can also actually create back injury and pain.

Other devices are passive in that the user positions himself or herself on the device and either the inherent shape of the device provides the stretch, or moving parts of the device stretch the back and spine. Although preferable over the active devices, these devices also suffer drawbacks. Many of the devices move parts into the spine causing discomfort. Others, are cushioned so as to conform to the body of the user, however, due to the wide variety of body types and sizes, the nonconforming cushioning can create undesirable and uncomfortable pressure points in many users' backs. Other moving devices are large, complicated, cumbersome and expensive. Non-moving devices, such as pillows and mattresses, which are contoured also suffer the drawbacks of not being able to conform to all body styles and shapes. There are yet other devices, such as fluid-filled spheres which do not create uncomfortable pressure points and conform to the user. Unfortunately, the pressure gradient intended to be applied to properly stretch and posture the spine is lost. Nearly all of the above devices fail to grant the user control over the degree of pressure or posturing applied.

The inventors of the present application invented the spinal therapy apparatus disclosed in U.S. Pat. No. 6,375,631. However, this apparatus is limited to a single form of therapy, mainly a person lying on his or her back and using the apparatus to apply an upward pressure gradient to the area to be treated. However, the apparatus is incapable of performing passive lumbar spinal extension, such as that performed during prone trunk extension exercises. Such exercises are typically performed lying prone on a flat surface, and lifting the head and torso upwardly and off of the ground. The exerciser typically lies on his or her stomach with the weight on his or her elbows or forearms, and the hips touching the floor or mat. The shoulders are pulled backwards and the chest upward in order to extend the spine and exercise the lumbar spinal extensor group, as well as the stabilizing muscles of the neck, obliques, and hips. Those exercisers with sufficient strength or fairly low back pain may perform such exercises by lying on their stomach with palms on the floor near the shoulders, as if performing a standard push-up.

While such exercises are beneficial to the individual as described above, many individuals are incapable of performing such strenuous exercise. Additionally, individuals performing such exercises may actually overexert themselves and create additional pain or even cause additional injury.

It has been found that using a fluid-filled pad, such as an air mattress, or even a foam mattress with such an apparatus as that disclosed in U.S. Pat. No. 6,375,631, or co-pending patent application Ser. No. 10/283,846 results in the pad or mattress being lifted and forming a sharp “V” shape. Such a posture does not provide a uniform pressure gradient across the intended area of the spine to be treated, but rather can unduly focus the pressure on a very small portion of the spine. In certain instances, this can be undesirable and perhaps even uncomfortable to the user of the spinal therapy apparatus. Ideally, the mattress or pad would curve or bow in response to being lifted by the spinal therapy apparatus, thus applying a graduating or more uniform pressure gradient across the area of the spine treated by the apparatus.

Accordingly, there is a need for an apparatus which passively treats the spine and applies a sufficient pressure gradient to the area to be treated without causing pressure point discomfort in the user's back. What is also needed is an apparatus that allows the individual to passively perform trunk stretching exercises. What is also needed is an apparatus which utilizes a mechanically created pressure gradient which is controlled by the user to treat the user's back. What is further needed is a reinforced mattress for use with such a spinal therapy apparatus. What is further needed is an apparatus which is uncomplicated, relatively inexpensive and easily stored. The present invention fulfills these needs and provides other related advantages.

### SUMMARY OF THE INVENTION

The present invention resides in a spinal therapy apparatus which passively treats the spine and applies sufficient pressure gradient to the area to be treated without causing pressure point discomfort in the user's back. The apparatus is generally comprised of a base assembly having a pair of spaced-apart, parallel guide posts, extending upwardly from a platform. The guide posts may be capable of being selectively pivoted between a first position and a second position. A lift bar assembly includes two lift bars slidably attached to the guide posts, and a cross-bar interconnected between the lift bars. The lift bars interiorly receive the guide posts. An elongated mattress is disposed between the guide posts and over at least a portion of the horizontal cross bar for support of the user. An actuator selectively raises and lowers the lift bar assembly relative to the base assembly, so as to apply a pressure gradient to the area to be treated.

The mattress includes at least one resiliently flexible slat such that while the lift bar assembly raises the mattress, the slap provides reinforcement of the mattress and causes the mattress to bow, thus applying a more uniform and graduating pressure gradient to the area of the spine to be treated. The at least one slat may be comprised of a plastic material having such resiliently flexible qualities. Preferably, the at least one slat comprises a plurality of slats in overlapping relation to one another. Typically, such slats are disposed over a central portion of the mattress. In a particularly preferred embodiment, at least two slats are disposed along a longitudinal axis of the mattress, and at least two additional slats are disposed generally transverse to the longitudinal slats.

In one embodiment, the mattress includes a cover having at least one pocket configured to house the one or more slats. Preferably, the pocket is configured to removably receive the slat. In accordance with the preferred embodiment, at least two pockets are disposed generally along the longitudinal axis of the mattress about a central portion thereof, with at least two additional pockets being disposed generally transverse to the longitudinal pockets to accept the slat arrangement described above.

In use, a user of the device lies on the mattress and actuates the motor to raise the lift bar assembly until a sufficient pressure gradient is applied to the area of the back to be treated. If the pressure gradient is exceeded, the user may actuate the motor to lower the lift bar assembly. Similarly, if additional pressure is desired, the user actuates the motor to raise the lift bar assembly. The user can position the area to be treated directly over the horizontal cross bar, or an area of the back adjacent to the area to be treated depending upon the level of discomfort and the aggressiveness of the therapy. In order to perform trunk extension exercises, the guide posts and lift bar assembly are pivoted to an angled position, as described above. The user places his or her chest on the mattress over the cross bar and actuates the motor to raise and lower the lift bar assembly.

Other features and advantages of the present invention will become apparent from the following more detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a perspective view of an apparatus for performing spinal therapy embodying the present invention, and having a cushion pad and user positioned over a cross bar thereof for performing a first spinal therapy exercise;

FIG. 2 is a perspective view of the apparatus of the present invention having a mattress and a user positioned over the cross bar for performing a second trunk extension exercise;

FIG. 3 is a perspective view of an apparatus of the present invention in a lowered state;

FIG. 4 is a perspective view of the apparatus of the present invention having a cross bar thereof in an elevated state;

FIG. 5 is an elevational view of a cable and pulley system used in accordance with the present invention to raise and lower a lift bar assembly of the present invention in a uniform manner;

FIG. 6 is an elevational view of the cable and pulley system positioned to raise the lift bar assembly;

FIG. 7 is a partially fragmented perspective view of the apparatus of the present invention;

FIG. 8 is an enlarged perspective view of area “8” of FIG. 7, illustrating a lift bar having a pulley therein, and connected to a rod of an actuator of the present invention;

FIG. 9 is an enlarged fragmented perspective view of area “9” of FIG. 7, illustrating an opposite lift bar disposed over a guide post, and having a cable of the pulley system attached thereto;

FIG. 10 is a side elevational view of the apparatus of the present invention, positioned to perform the first spine therapy exercise;

FIG. 11 is a side elevational view of the apparatus of the present invention positioned at an angle so as to perform the second trunk stretching exercise of the present invention;

FIG. 12 is a perspective view of the apparatus of the present invention positioned at an angle to perform the trunk stretching exercise;

FIG. 13 is a perspective view of the apparatus of the present invention;

FIG. 14 is an enlarged perspective view of area “14”, illustrating a lift bar assembly thereof slightly raised;

FIG. 15 is a perspective view of the apparatus of the present invention disposed at a pivoted position;

FIG. 16 is an enlarged perspective view of area “16” of FIG. 15, showing a brace thereof in an extended position and in contact with a platform stop, in accordance with the present invention;

FIG. 17 is an enlarged perspective view similar to FIG. 16, illustrated a second brace extended into contact with the platform stop to further pivot the lift bar assembly;

FIG. 18 is an exploded perspective view of various components comprising the apparatus of the present invention, illustrating their inter-relation and connecting parts;

FIG. 19 is a perspective view of a reinforced mattress embodying the present invention overlying a lift bar assembly of the apparatus, shown in phantom;

FIG. 20 is a perspective view taken generally along line 20-20, illustrating an arrangement of slats associated with the mattress; and

FIG. 21 is a partially exploded perspective view illustrating the slats insertable into pockets of a mattress cover.

### DETAILED DESCRIPTION OF THE PREFERRED EMBODIMENTS

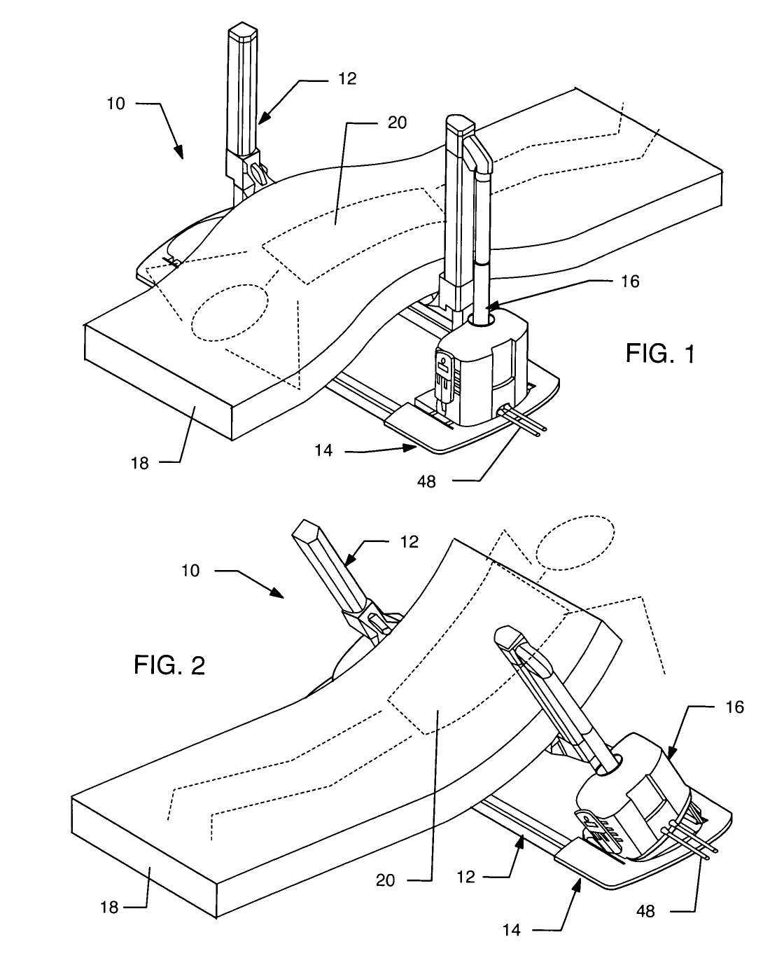

As shown in the drawings for purposes of illustration, the present invention is concerned with a spinal therapy apparatus, generally referred to by the reference number 10. The apparatus 10 is generally comprised of a lift bar assembly 12 which is slidably positioned on a base assembly 14 in response to an actuator 16 which is connected to the lift bar assembly 12. A cushioned pad or mattress 18 is positioned over the lift bar assembly 12, and in one exercise of the present invention, the user 20 lies on his or her back on the pad 18 so that the spine is generally over the lift bar assembly 12. As the lift bar assembly 12 is elevated, as illustrated in FIG. 1, vertical lifting action across a portion of the users spine passively exercises and stretches the spine and back. A second trunk stretching exercise can be performed, as illustrated in FIG. 2, by tilting the lift bar assembly 12 and positioning the user's chest over the lift bar assembly 12 so that the head and torso are raised at an angle so as to passively exercise and stretch the spine and back in a different manner.

Use of the present invention as described herein, passively mobilizes the spine to obtain normal lumbar spine kinematics and ROM in all planes. The continuous passive motion of the lumber spine provides joint nutrition and stress reduction. Use of the apparatus 10 of the present invention also stretches the user's back and provides core strengthening and stabilization in order to restore function and eliminate stress and pain.

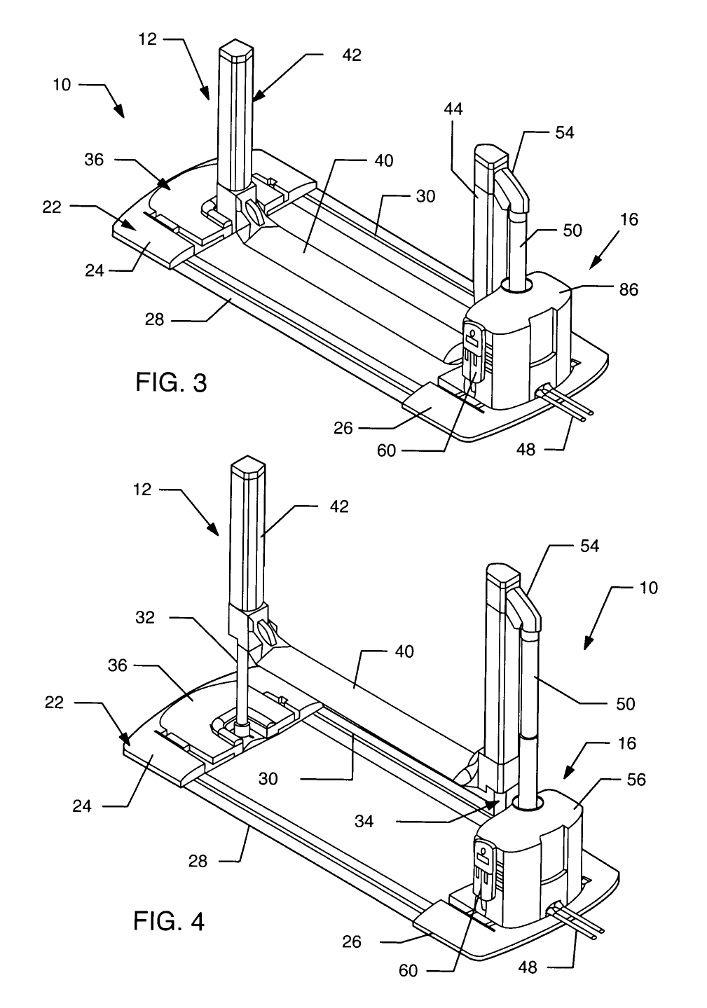

With reference now to FIGS. 3, 4 and 18, The base assembly 14 is generally comprised of a support platform 22 having two mounting plates 24 and 26 interconnected by platform legs 28 and 30 which are relatively flat and of low profile. The mounting plate 24 and 26 and connecting platform legs 28 and 30 provide a stable base for the apparatus 10.

Guide posts 32 and 34 extend upwardly from the platform 22 so as to be spaced-apart from one another and generally parallel. The guide posts 32 and 34 each include a base 36 and 38 at a lower end thereof which is pivotly attached to the mounting plates 24 and 36, respectively. As will be more fully described herein, the pivotal connection of the guide post bases 36 and 38 to the platform 22 allow the guide posts 32 and 34 and the lift bar assembly 12 to be moved from a generally vertical position to an angled position in order to allow the user 20 to perform passive trunk or lumbar stretch exercises, as illustrated in FIG. 2.

The lift bar assembly 12 is slidably attached to the guide posts 32 and 34. The lift bar assembly 12 includes a horizontal cross bar 40 interconnected between two lift bars 42 and 44 which are configured to interiorly receive the guide posts 32 and 34.

As shown in the various drawings, in particular FIGS. 3 and 4, the actuator 16 moves the lift bar assembly upwardly and downwardly. The actuator 16 includes a motor 46 which is preferably powered by electrical leads 48 extending to an electrical outlet (not shown). A guide tube 50 is connected to the motor and telescopically receives a rod 52 or the like which is operably connected to the motor 46 so as to extend or retract by pneumatic means, a screw mechanism, or in any other suitable fashion. A cap 54 is configured to be securely placed over a top end of lift bar 44. The cap 54 includes an extension which is configured to receive the extendable rod 52. Thus, as rod 52 is extended upwardly, the lift bar 44, and lift bar assembly 12, is also raised. Similarly, when the rod 52 is gradually lowered, the lift bar assembly 12 is lowered in a similar fashion.

A housing 56 encases the motor 46, and is attached to guide post base 38 by appropriate means, for example the illustrated mounting ears 58 for snap-fit connection or connection with a pin or the like. Thus, as the guide post base is pivoted, the actuator 16 is also pivoted. In a particularly preferred embodiment, an electronic hand-held remote 60 is used to activate the motor 46 and raise or lower the lift bar assembly 12 in a selective manner.

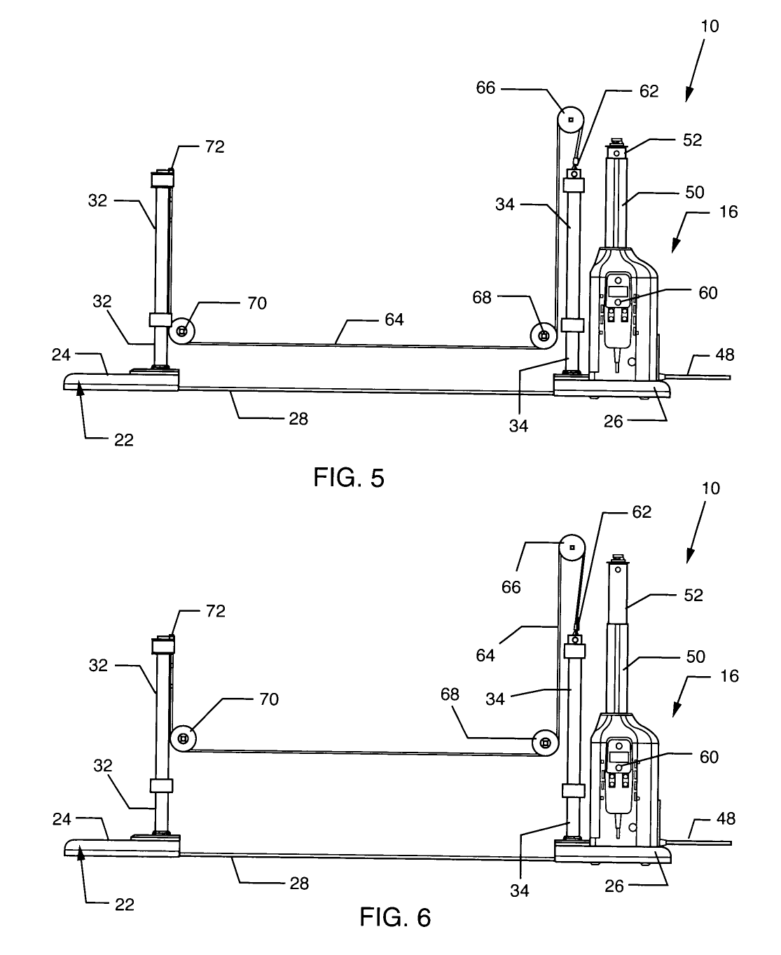

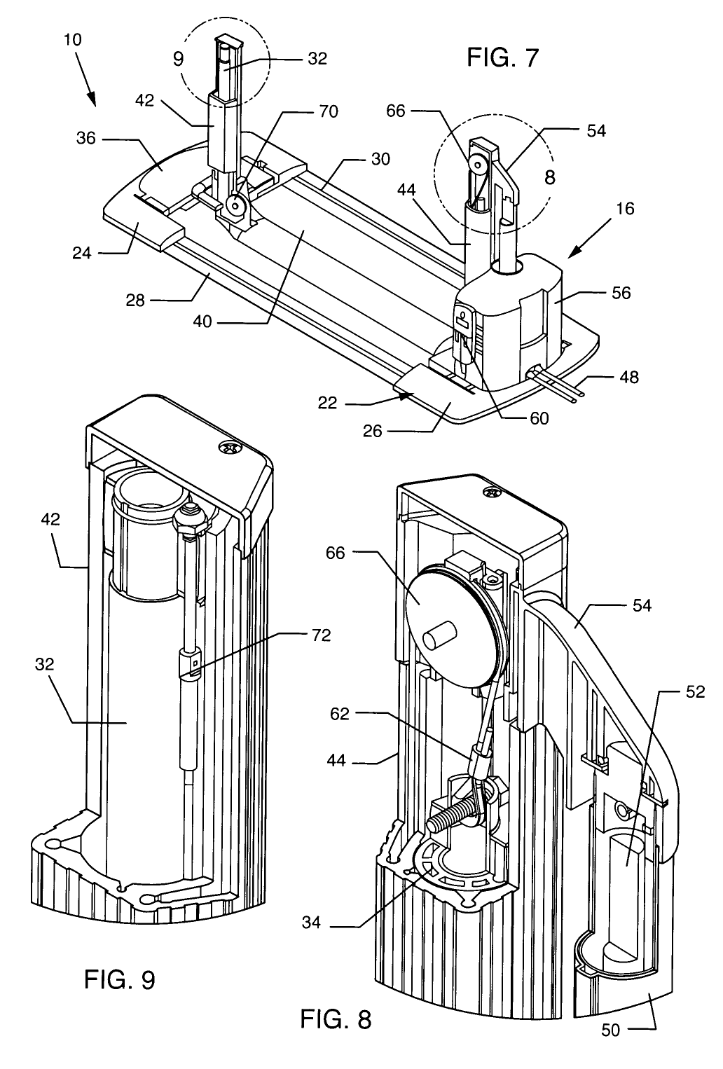

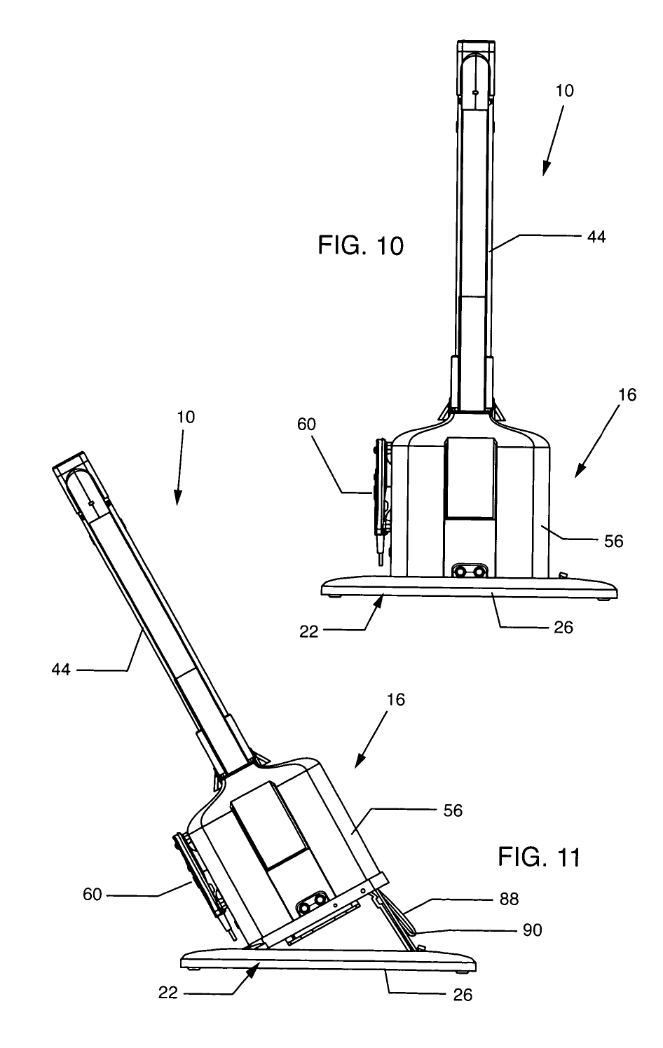

In order to keep the entire lift bar assembly 12 parallel to the guide posts 22 and 34 and also to prevent binding, a cable and pulley system is employed. With reference to FIGS. 5-9, a first end 62 of a cable 64 is attached to a top end of the guide post 32. The cable loops over a pulley 66, typically housed in the cap 54 or in an upper portion of lift bar 44. Cable 64 extends over pulley 66, through cap 54 and through the length of the lift bar 44 through pulley 68 which is disposed at the junction between the lift bar 44 and cross bar 40. The cable 64 extends from its engagement with pulley 68 under or through horizontal cross bar 40 to pulley 70 at the opposite end of the cross bar 40. The cable 64 extends upwardly through lift bar 42 to a connection point 72 at the top of guide post 32. Thus, as the motor 46 is activated and rod 52 extends upwardly, pulley 66 creates a loop of cable 64 which effectively shortens the overall length of the cable 64 over its path and causes the lower pulleys 68 and 70 to raise the entire lift bar assembly 12 in a uniform manner. Without the use of the pulley system, extending rod 52 would cause lift bar 44 to rise, at the risk of binding lift bar 42.

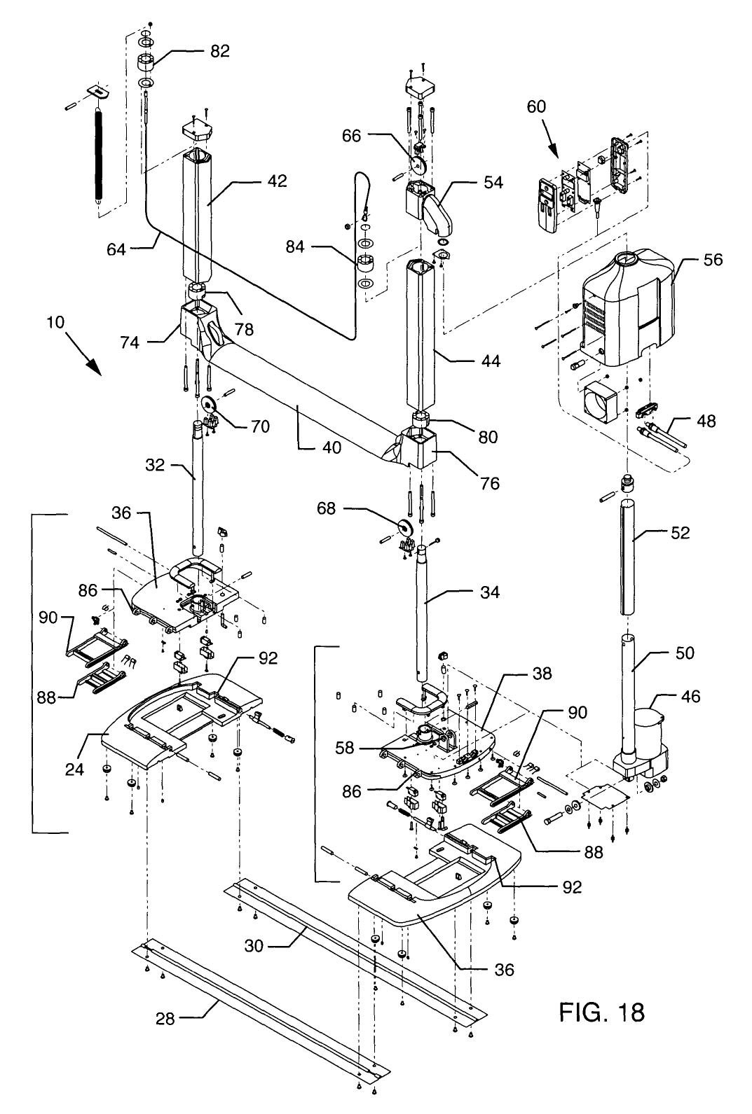

With reference now to FIG. 18, the cross bar 40 is typically attached to the lift bars 42 and 44 by means of elbows 74 and 76. The elbows 74 and 76 are attached to the base of each lift bar 42 and 44, and the horizontal cross bar is attached to the elbows 74 and 76 by any appropriate means. Typically, bearings 78 and 80 are secured in pockets of elbows 74 and 76 to facilitate sliding relationship between the lift bars 42 and 44 and guide posts 32 and 34. Thus, inner surfaces of bearings 72 and 80 slide along the outside surface of the guide posts 32 and 34. The bearings 78 and 80 are also configured such that upon encountering stops 82 and 84 attached to an upper portion of guide posts 32 and 34, upward motion of the lift bar is restricted. In a particularly preferred embodiment, the outer surfaces of the stops 82 and 84 slidably engage the inner surfaces of the lift bars 42 and 44 provide a stable and fluid motion while the lift bar assembly 12 is raised and lowered.

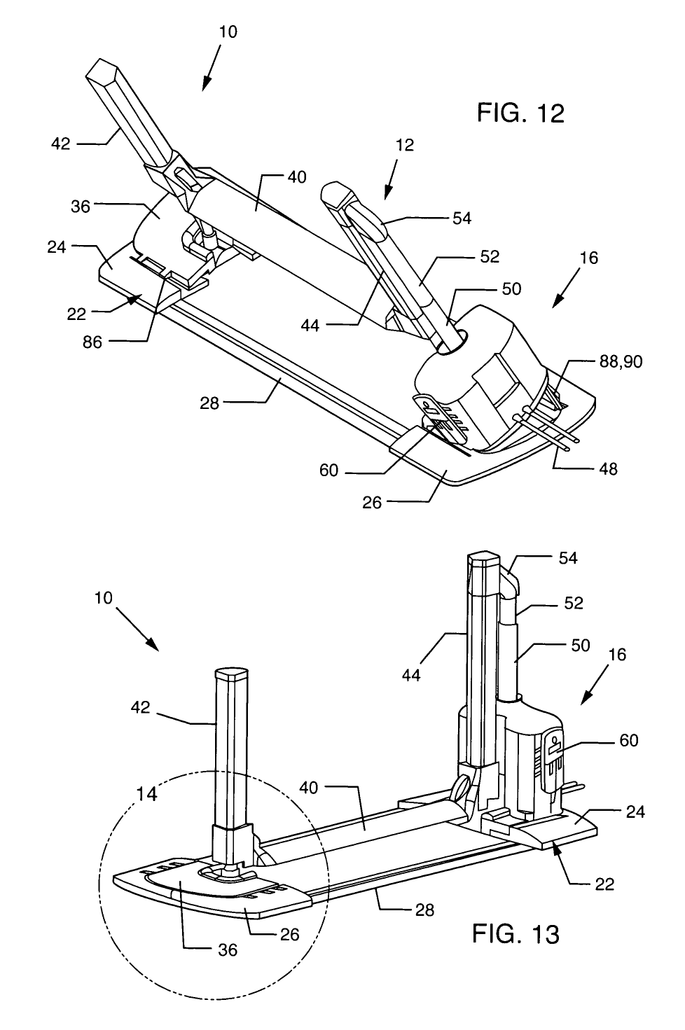

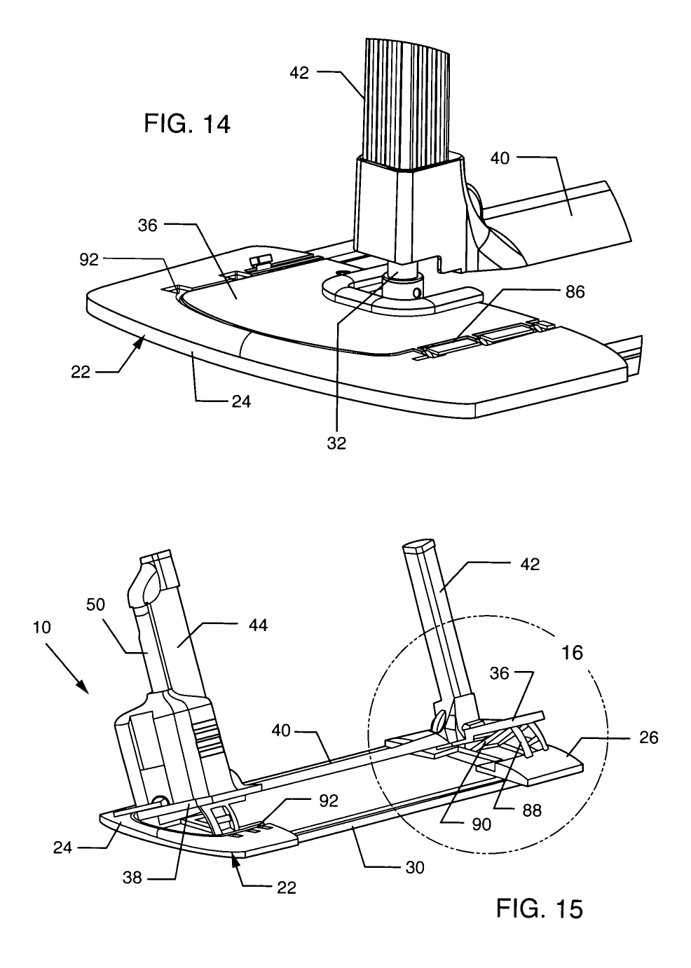

With reference now to FIGS. 10-16, the apparatus 10 can be moved into different positions for different passive exercises. As illustrated in FIG. 10, the guide posts 32 and 34 and lift bar assembly 12, can be positioned in a generally vertically orientation such that the horizontal cross bar 40 can serve to lift the spine of the user lying on his or her back as illustrated in FIG. 1. However, in order to perform a trunk stretch or lumbar spinal extension exercise, the guide posts 32 and 34, actuator 16, and lift bar assembly 12 are pivoted to a second positioned angled from vertical, as illustrated in FIG. 11. In this position, the cross bar 40 lifts the upper torso of the user 20, who is lying prone over the apparatus 10, upwardly at an angle, as shown in FIG. 2.

As mentioned above, each guide post base 36 and 38 is pivotly attached to the mounting plate 24 and 26, such as by a hinge 86 or the like. Braces 88 and 90 are pivotly attached to an underside of the guide post base 36 and 48 so as to fold under the guide post base 36 and 38 when the guide posts 32 and 34 are vertically positioned. The braces 88 and 90 are capable of extending outward, as shown in FIGS. 15-17, to place the aforementioned components at an angle. Typically, the mounting plates 24 and 26 of the platform 22 include depressions 92 or the like formed therein which are configured to receive the ends of the braces 88 and 90 and serve as a stop.

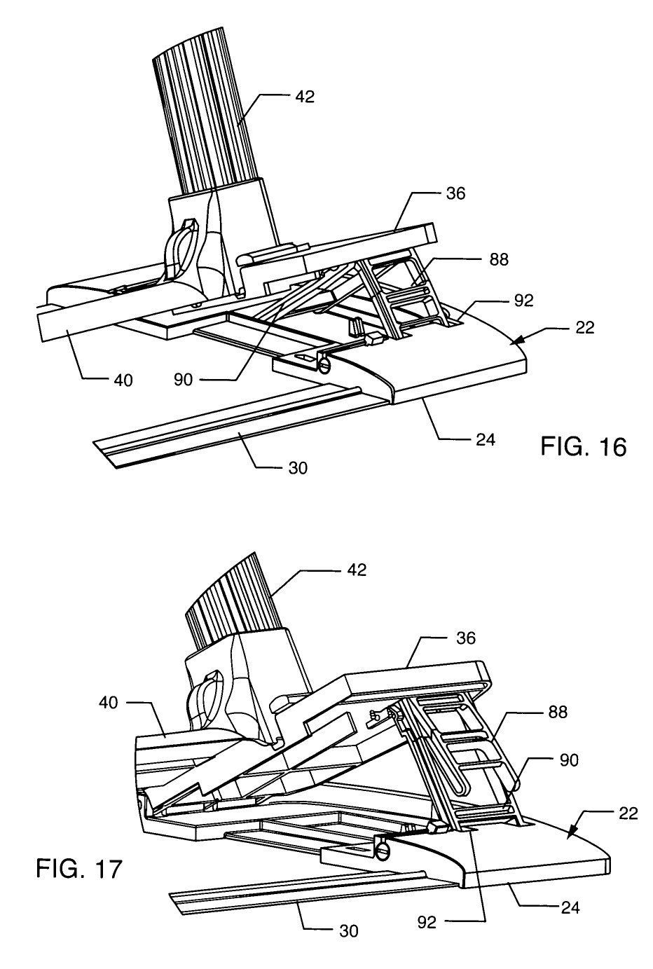

In a particularly preferred embodiment, the braces 88 and 90 are of differing lengths so that user may position guide post bases 36 and 38, and thus guide posts 33 and 34, actuator 16 and lift bar assembly 12, at differing angles. Increasing the angle from vertical provides a more rigorous stretch and exercise. Preferably, as illustrated in FIG. 17, the braces 88 and 90 are pivotly connected to the guide post base 36 or 38 at approximately the same location. The longer brace 90 can be kept folded under the base 36 or 38 as illustrated in FIG. 16, with the shorter brace inserted into the platform stops 92. Pulling the lift bar assembly 12 back further unfolds the larger brace 90 and lifts the shorter brace 88 from the stops 92 until the ends of brace 90 are positioned within stops 92 to secure the apparatus 10 at the desired position.

The important aspect of the present invention is that the lift bar assembly 12 and guide posts 32 and 34 be selectively angled from vertical in order to allow the user to passively perform trunk stretching exercises, as illustrated in FIG. 2. Thus, different structural designs accomplishing the same purpose are intended to fall within the scope of the present invention. As such, the brackets 88 and 90 could feasibly be directly attached to the guide posts 32 and 34. Those skilled in the art will appreciate other design alternatives not fully described herein.

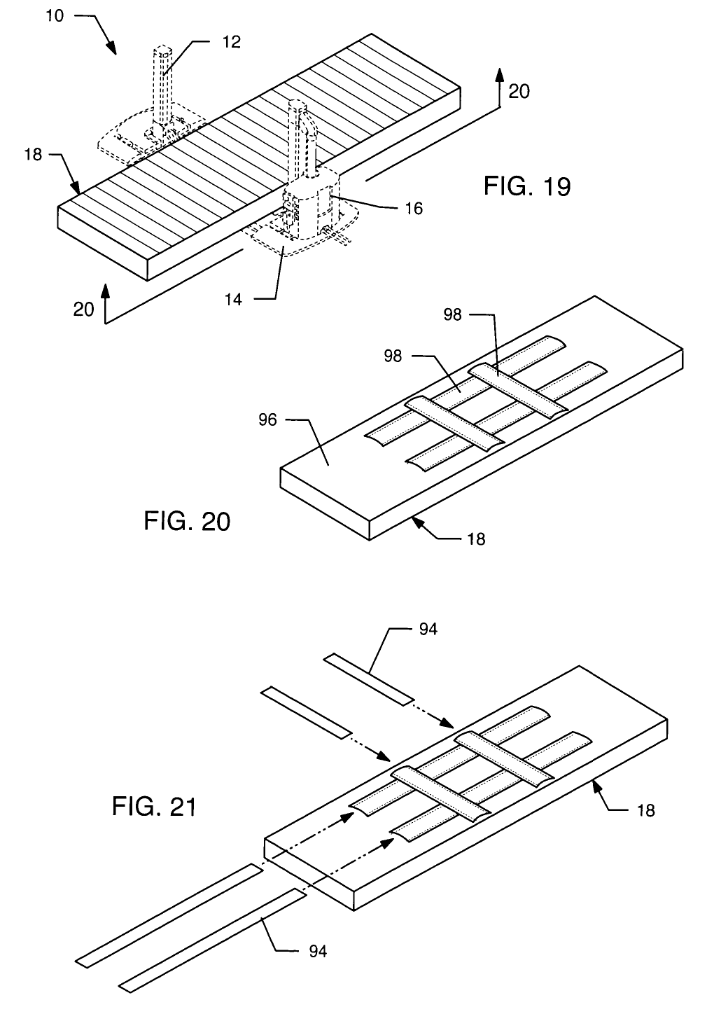

With reference now to FIGS. 19-21, in a particularly preferred embodiment, the mattress 18 is reinforced such that upon being lifted upwardly by the lift bar assembly 12, the mattress 18 bows or curves to apply a more gradual and uniform pressure gradient to the user of the apparatus 10, as illustrated in FIGS. 1 and 2. The mattress 18 may be comprised of any suitable material, such as foam or the like, or even an air-filled mattress.

As illustrated in FIGS. 20 and 21, the mattress 18 has at least one, and preferably a plurality of slats 94 which are associated with the mattress 18 to provide reinforcement thereto. The slats 94 are comprised of a material which is resiliently flexible, such as plastic, such that as the mattress 18 is lifted by the lift assembly 12, the slats 94 impose a bowed or curved shape to the mattress 18, yet return to a generally flat state upon lowering of the assembly 12. It has been found that using a plurality of slats 94 in overlapping relation over a central portion of the mattress 18 provides the best results. A particularly preferred arrangement of slats 94 comprises at least two slats 94 being directed along the longitudinal axis of the mattress 18, with at least two additional slats 94 disposed generally transverse to the longitudinal slats. Such an arrangement provides the curved and bowed shape of the mattress 18 as described above.

In a particularly preferred embodiment, the mattress 18 is encased within a cover 96, such as a cloth cover which can be laundered. The cover 96 includes pockets 98 which are sized and configured such so as to receive the slats 94. Preferably, the pockets 98 are arranged so as to be over a central portion of the mattress 18 and accept the configuration described above, namely, two longitudinal slats 94, with at least two additional slats 94 being disposed generally transverse to the longitudinal slats 94. Typically, the pockets 98 allow for the removal of the slats 94, as illustrated in FIG. 21, so that the cover 96 can be laundered, the mattress 18 more easily stored, etc.

Although the mattress 18 has been illustrated and described as using a cover 96 having pockets 98 for receipt of slats 94 as a particularly preferred embodiment, it should be understood that the slats 94 can be attached directly to the mattress 18, or even embedded within the mattress 18 so long as the least one or more slat 94 provides the curved or bowed effect and the benefits thereof as described above.

In use, and with reference to FIG. 1, the mattress 18 is positioned over the horizontal cross bar 40 between the guide posts 32 and 34 to provide cushion support for the user's back. The user 20 positions the portion of the back to be treated, typically the lower or lumbar section of the spine, over the horizontal cross bar 40 and activates actuator 16 to raise the lift bar assembly 12. This is typically done by using the remote control device 60 electrically connected to motor 46. The actuator 16 raises or lowers the rod 52, and thus the lift bar assembly 12, providing sufficient pressure gradient to the targeted location of the spine of the user. The user 20 can relax during the exercise which aids in the spinal therapy as muscle tension and tightening is eliminated, allowing the vertebrae to hyper-extend and stretch more easily to eliminate the vertebrae and decompression related pain.

Similar benefits are provided in a different manner and to different muscle groups by positioning the apparatus 10 in the angled position, as described above, in order to allow the user to lie prone over the cross bar 40. As the lift bar assembly 12 is elevated at an angle, the user's chest is lifted upwardly at an angle to passively perform a trunk stretch or lumbar spine extension. Those of very little strength are able to perform the exercises of the present invention due to the motorized nature of the apparatus 10. Also, the user 20 is able to selectively control the degree of lift of the lift bar assembly 12 to accommodate his or her physical condition.

Although several embodiments of the present invention have been described in detail for purposes of illustration, various modifications of each may be made without departing from the spirit and scope of the invention. Accordingly, the invention is not to be limited, except as by the appended claims.

What is claimed is:

1. A spinal therapy apparatus, comprising:
an apparatus including a lift bar assembly slidably attached to parallel guide posts and an actuator for raising and lowering the lift bar assembly; and
a mattress configured to overlay at least a portion of the lift bar assembly between the guide posts and having at least one resiliently flexible slat associated therewith, whereby the at least one slat provides reinforcement to the mattress and causes the mattress to bow while the lift bar assembly is raised.

2. The mattress of claim 1, wherein the at least one slat is comprised of a plastic material.

3. The mattress of claim 1, wherein the at least one slat comprise at least two slats disposed along a longitudinal axis of the mattress.

4. The mattress of claim 3, wherein the slats further comprise at least two slats disposed generally transverse to the longitudinal slats.

5. The mattress of claim 1, wherein the mattress includes a cover having at least one pocket configured to house the at least one slat.

6. The mattress of claim 5, wherein the at least one pocket is configured to removably receive the at least one slat.

7. The mattress of claim 5, wherein the at least one pocket comprises at least two pockets disposed generally along a longitudinal axis of the mattress, and at least two pockets disposed generally transverse to the longitudinal pockets.

8. The mattress of claim 1, wherein the at least one slat is associated with a central portion of the mattress.

9. The mattress of claim 8, wherein the at least one slat comprises a plurality of slats in overlapping relation.

10. A spinal therapy apparatus, comprising:
a spinal therapy apparatus including a lift bar assembly slidably attached to parallel guide posts and an actuator for raising and lowering the lift bar assembly; and
a mattress configured to overlay at least a portion of the lift bar assembly between the guide posts having a plurality of resiliently flexible slats associated with a central portion thereof in overlapping relation, whereby the slats provide reinforcement to the mattress and cause the mattress to bow while the lift bar assembly is raised.

11. The mattress of claim 10, wherein the slats are comprised of a plastic material.

12. The mattress of claim 10, wherein the slats comprise at least two slats disposed along a longitudinal axis of the mattress, and at least two slats disposed generally transverse to the longitudinal slats.

13. The mattress of claim 10, wherein the mattress includes a cover having pockets configured to house the slats.

14. The mattress of claim 13, wherein the pockets are configured to removably receive the slats.

15. The mattress of claim 13, wherein the pockets comprise at least two pockets disposed generally along a longitudinal axis of the mattress, and at least two pockets disposed generally transverse to the longitudinal pockets.

16. A spinal therapy apparatus, comprising:
a spinal therapy apparatus including a lift bar assembly slidably attached to parallel guide posts and an actuator for raising and lowering the lift bar assembly; and
a mattress configured to overlay at least a portion of the lift bar assembly between the guide posts, the mattress having a plurality of resiliently flexible slats associated therewith, wherein the slats comprise at least two slats disposed along a longitudinal axis of the mattress, and at least two slats disposed generally transverse and in overlapping relation to the longitudinal slats;
whereby the slats provide reinforcement to the mattress and cause the mattress to bow while the lift bar assembly is raised.

17. The mattress of claim 16, wherein the slats are comprised of a plastic material.

18. The mattress of claim 16, wherein the mattress includes a cover having pockets configured to house the slats.

19. The mattress of claim 18, wherein the pockets are configured to removably receive the slats.

20. A spinal therapy apparatus, comprising:
an apparatus including a lift bar assembly slidably attached to parallel guide posts and an actuator for raising and lowering the lift bar assembly; and
a mattress configured to overlay at least a portion of the lift bar assembly between the guide posts;
a plurality of resiliently flexible slats associated with the mattress; and
a mattress cover having pockets configured to house the slats;
whereby the slats provide reinforcement to the mattress and cause the mattress to bow while the lift bar assembly is raised.

21. The mattress of claim 20, wherein the slats are comprised of a plastic material.

22. The mattress of claim 20, wherein the pockets are configured to removably receive the slats.

23. The mattress of claim 20, wherein the pockets comprise at least two pockets disposed generally along a longitudinal axis of the mattress, and at least two pockets disposed generally transverse to the longitudinal pockets.

24. The mattress of claim 20, wherein the slats are associated with a central portion of the mattress.
